(19) United States
(12) Patent Application Publication
(10) Pub. No.: US 2022/0378349 A1
(43) Pub. Date: Dec. 1, 2022

(54) A NOVEL MEANS OF ASSESSING MUSCULAR FUNCTION AND FRAILTY

(71) Applicant: ARIZONA BOARD OF REGENTS ON BEHALF OF THE UNIVERSITY OF ARIZONA, Tucson, AZ (US)

(72) Inventors: Marvin J. Slepian, Tucson, AZ (US); Kaitlyn Ammann, Tucson, AZ (US); Yonathan Weiner, Worcester, MA (US)

(21) Appl. No.: 17/769,974

(22) PCT Filed: Oct. 19, 2020
(86) PCT No.: PCT/US20/56343

§ 371 Date: Apr. 18, 2022

Related U.S. Application Data
(60) Provisional application No. 62/923,090, filed on Oct. 18, 2019.

(51) Int. Cl. A61B 5/22; G01P 13/00; A61B 5/397; A61B 5/11; A61B 5/00

(52) U.S. Cl. CPC A61B 5/224; G01P 13/00; A61B 5/397; A61B 5/1122; A61B 5/7267; A61B 5/7282; A61B 5/1124; A61B 2562/0219; A61B 2562/164; A61B 2562/066

(57) ABSTRACT

The present invention is directed to a device for measuring muscular fatigue through obtaining a signal generated by repetitive motions of a human body. The present invention features a system for measuring movements of one or more muscles of a body of a user in order to track muscular fatigue. The system may comprise an object. The system may further comprise a motion sensor configured to measure signals in response to a stimulation and transmit said signal to a computing device. The stimulation may comprise an action carried out repetitively over an interval of time. The motion sensor is configured to measure a progressive reduction in a signal over the repetitive motions and disposed relative to the one or more muscles of the body of the user. The computing device may be capable of measuring muscular fatigue by measuring parameters of the received signals and generating a muscular fatigue signal.

### CROSS-REFERENCES TO RELATED APPLICATIONS

This application claims benefit of U.S. Provisional Application No. 62/923,090 filed Oct. 18, 2019, the specification of which is incorporated herein in their entirety by reference.

### FIELD OF THE INVENTION

The present invention is directed to a device for measuring muscular function and fatigue through obtaining a signal generated by continuous repetitive motions of a human body.

### BACKGROUND OF THE INVENTION

With more than 14% of the population now, which is predicted to increase to 20% in 20 years, over 65 years old in the United States, geriatric care needs increasing attention as the number of older adults grows rapidly. Older adults suffer from low physio-logic reserve as they age and are more vulnerable to poor outcomes, such as longer length of hospital stay, higher rate of surgical complications, and increased risk of disability, institutionalization, and death. The highly prevalent frailty among older adults is considered significantly useful in identifying these hemeostenotic older adults and therefore in decision-making for interventions. A cycle of frailty consists of many factors of clinical signs and symptoms with some core elements such as sarcopenia.

Despite the significant pathophysiologic and prognostic role of frailty, no simple and fast approaches to identify and assess frailty exist. Current clinical setups are normally bulky and time consuming or not applicable for mobility-impaired non-ambulatory patients. An assessment method for upper-extremity frailty (UEF) utilizing low cost sensors was developed and validated using the Fried index. While this method shows satisfactory 78% sensitivity and 82% specificity of predicting the frailty status, it requires large movement of the upper arm, which would limit the applicability to patients with upper-extremity disability or injury.

Conversely, compressing a ball or other object requires very minimal space to perform. This exercise involves both isometric and isotonic muscular activity. A dense and stiff ball, i.e., non-elastomeric, requires an isometric muscle contraction of the forearm to squeeze it. In contrast, compressing a soft and elastomeric ball then would result in isotonic contraction, in that the muscle will both contract and change its length. Consecutive gripping or compressing motion would lead to reduced strength and lowered speed, As previous studies demonstrated that slowness and weakness of movements are markers of frailty, frailty can be measured and determined by muscle fatigue of upper extremity. As such, it is hypothesized that a range of performance standards for individuals based on age, nutritional status, and muscular conditioning exists for people conducting this ball compression assessment isotonically and isometrically. Similarly, with progressive aging, mylopenia/sarcopenia, and other frailty or diseased states individuals may have radically differing levels of performance. Thus, there exists a present need for a wearable wireless motion sensor with embedded surface (EMG) electrodes capable of sufficiently assessing fatigue against varying muscle loads.

### BRIEF SUMMARY OF THE INVENTION

It is an objective of the present invention to provide devices, systems, and methods that allow for measuring muscular function and fatigue through obtaining a signal generated by continuous repetitive motions of a human body, as specified in the independent claims. Embodiments of the invention are given in the dependent claims. Embodiments of the present invention can be freely combined with each other if they are not mutually exclusive.

The present invention features a motion sensor for measuring movements of one or more muscles of a body of a user in order to track muscular fatigue. In some embodiments, the motion sensor may comprise an electromyogram (EMG) for measuring an electrical activity in response to a stimulation to generate an EMG signal, The motion sensor may further comprise a gyroscope for measuring an angular velocity in response to the stimulation in order to generate an angular velocity signal. The motion sensor may further comprise an accelerometer for measuring an acceleration in response to the stimulation, and an orientation of the motion sensor and the one or more muscles of the body of the user in relation to gravity to generate an acceleration signal and an orientation signal, respectively. The motion sensor may further comprise a transmission component for transmitting the EMG signal, the angular velocity signal, the acceleration signal, and the orientation signal to a computing device. The stimulation may comprise an action carried out repetitively over an interval of time. In some embodiments, this action may be squeezing an object in a hand, and squeezing the object between thighs, or moving a back against the object. The motion sensor is configured to measure a progressive reduction in a signal over the repetitive motions. The motion sensor may be disposed relative to the one or more muscles of the body of the user. The computing device may be capable of measuring muscular fatigue by measuring various parameters of the received signals.

The present invention features a system for measuring movements of one or more muscles of a body of a user in order to track muscular fatigue. In some embodiments, the system may comprise an object. The system may further comprise a motion sensor comprising an EMG for measuring an electrical activity in order to generate an EMG signal, a gyroscope for measuring an angular velocity to generate an angular velocity signal, and an accelerometer for measuring an acceleration and an orientation of the motion sensor and the one or more muscles of the body of the user in relation to gravity to generate an acceleration signal and an orientation signal, respectively. The motion sensor may further comprise a transmission component for transmitting the EMG signal, the angular velocity signal, the acceleration signal, and the orientation signal to a computing device. The stimulation may comprise an action carried out repetitively over an interval of time. In some embodiments, this action may be squeezing an object in a hand, and squeezing the object between thighs, or moving a back against the object. The motion sensor is configured to measure a progressive reduction in a signal over the repetitive motions. The motion sensor may be disposed relative to the one or more muscles of the body of the user. The computing device may be capable of measuring muscular fatigue by measuring various parameters of the received signals. The system may further comprise a computing device comprising a display component. The computing device may further comprise a memory component comprising instructions for generating a muscular fatigue signal by measuring various parameters. The memory component may further comprise instructions for displaying the muscular fatigue signal on the display component. The computing device may further comprise a processor capable of executing the computer-readable instructions stored on the memory component. The computing device may further comprise a receiver component for receiving the EMG signal, the angular velocity signal, the acceleration signal, and the orientation signal from the motion sensor.

The present invention features a method for measuring movements of one or more muscles of a body of a user in order to track muscular fatigue. In some embodiments, the method may comprise attaching a motion sensor to an area of the body of the user relative to the one or more muscles, and executing a stimulation of the one or more muscles of the body of the user. In some embodiments, the stimulation may comprise an action carried out repetitively over an interval of time. The action may be squeezing an object in a hand, squeezing the object between thighs, or moving a back against the object. The motion sensor is configured to measure a progressive reduction in a signal over the repetitive motions. The method may further comprise using an EMG of the motion sensor to measure an electrical activity to generate an EMG signal. The method may further comprise using a gyroscope of the motion sensor to measure an angular velocity to generate an angular velocity signal. The method may further comprise using an accelerometer of the motion sensor to measure an acceleration, and an orientation of the motion sensor and the one or more muscles of the body of the user in relation to gravity to generate an acceleration signal and an orientation signal, respectively. The method may further comprise transmitting the EMG signal, the angular velocity signal, the acceleration signal, and the orientation signal to a computing device. The method may further comprise calculating a muscle fatigue signal by measuring various parameters. The method may further comprise displaying the muscle fatigue signal on a display component.

One of the unique and inventive technical features of the present invention is the use of a wearable sensor attached to a user by adhesive to record various changes in muscle activity over repeated actions to measure muscle deficiency. Without wishing to limit the invention to any theory or mechanism, it is believed that the technical feature of the present invention advantageously provides for a time and cost efficient way to measure muscle deficiency reliably in a patient without the need for internal scans or large and expensive equipment. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a motion sensor for measuring muscle fatigue of the present invention.

FIG. 2A shows a system for measuring muscle fatigue of the present invention. FIG. 2B shows a visual signal generated by the system of FIG. 2A. FIG. 2C shows an EMG signal generated by the system of FIG. 2A.

FIG. 3 shows a flow chart of a method for measuring muscle fatigue of the present invention.

FIG. 4A shows a series of squeezable balls that could act as the object of the system of the present invention. FIG. 4B shows a table of the series of squeezable balls of FIG. 4A mapped to their stiffness. FIG. 4C shows graph mapping force exerted on each squeezable ball to displacement caused as a result of said force.

FIG. 5A shows an EMG signal generated by squeezing and releasing a Spalding bouncing ball repeatedly over a first set of 5 seconds. FIG. 5B shows an EMG signal generated by squeezing and releasing the Spalding bouncing ball repeatedly over a second set of 5 seconds. FIG. 5C shows an EMG signal generated by squeezing and releasing the Spalding bouncing ball repeatedly over a third set of 5 seconds. FIG. 5D shows a graph comparing a change in time between repetitions of squeezing between the Spalding bouncing ball and a baseball.

FIG. 6A shows an EMG signal generated by squeezing and releasing a Spalding bouncing ball repeatedly over a first set of 5 seconds. FIG. 6B shows a visual signal generated by squeezing and releasing the Spalding bouncing ball repeatedly over a second set of 5 seconds. FIG, 6C shows a visual signal generated by squeezing and releasing the Spalding bouncing ball repeatedly over a third set of 5 seconds. FIG. 6D shows a graph comparing a change in displacement over repetitions of squeezing between the Spalding bouncing ball and a baseball.

FIG. 7 shows a diagram of muscle activity over a contraction of said muscle.

FIG. 8 shows a flow chart of an experimentation method of the present invention.

FIG. 9A shows a top view of a motion sensor of the present invention. FIG. 9B shows a bottom view of the motion sensor of the present invention. FIG. 9C shows a graph of tri-axial acceleration data recorded by the motion sensor during muscle compression. FIG. 9D shows a graph of electromyogram measurements during muscle compression.

FIG. 10A shows top view of a visual tracking simulation of ball compression from an experimental method of the present invention, FIG, 10B shows a side view of the visual tracking simulation of ball compression from the experimental method of the present invention. FIG. 10C shows a calculation of displacement of the ball as change in compression along a single axis. FIG. 10D shows a visual signal generated by a visual tracking system.

### DETAILED DESCRIPTION OF THE INVENTION

Following is a list of elements corresponding to a particular element referred to herein:

100 motion sensor

101 electromyogram

102 accelerometer

103 gyroscope

104 transmission component

200 object

300 computing device

301 receiver component

302 memory component

303 processor

304 display component

400 stretchable interconnects

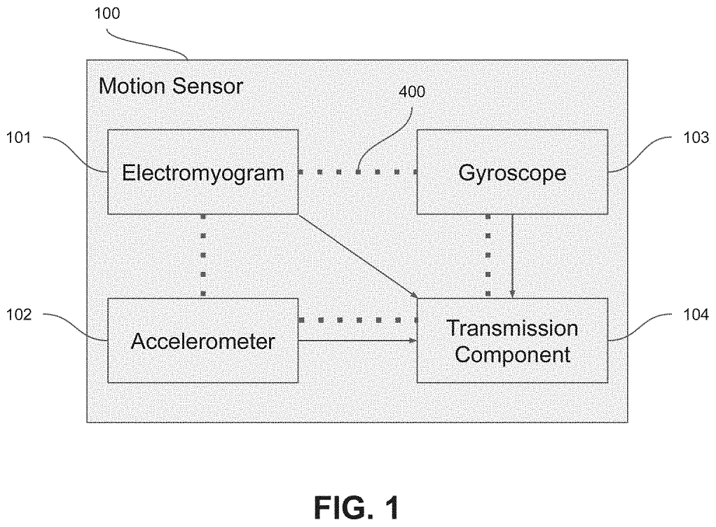

Referring now to FIG. 1, the present invention features a motion sensor (100) for measuring movements of one or more muscles of a body of a user in order to track muscular fatigue. In some embodiments, the motion sensor (100) may comprise an electromyogram (EMG) (101) for measuring an electrical activity in response to a stimulation of the one or more muscles of the body of the user in order to generate an EMG signal. The motion sensor (100) may further comprise a gyroscope (103) for measuring an angular velocity of the motion sensor (100) and the one or more muscles of the body of the user in response to the stimulation in order to generate an angular velocity signal. The motion sensor (100) may further comprise an accelerometer (102) for measuring an acceleration of the motion sensor (100) and the one or more muscles of the body of the user in response to the stimulation, and an orientation of the motion sensor (100) and the one or more muscles of the body of the user in relation to gravity to generate an acceleration signal and an orientation signal, respectively. The motion sensor (100) may further comprise a transmission component (104) for transmitting the EMG signal, the angular velocity signal, the acceleration signal, and the orientation signal to a computing device (300). The motion sensor (100) may further comprise a plurality of stretchable interconnects. Each stretchable interconnect (400) may extend between two components to connect said two components in order to facilitate conformal attachment to the subject. The stimulation may comprise an action carried out repetitively over an interval of time. In some embodiments, this action may be squeezing an object (200) in a hand, and squeezing the object (200) between thighs, or moving a back against the object (200). In other embodiments, this action may be lifting a weight, or moving a head. The motion sensor (100) is configured to measure a progressive reduction in a signal over the repetitive motions. The motion sensor (100) may be disposed relative to the one or more muscles of the body of the user. The computing device (300) may be capable of measuring muscular fatigue by measuring various parameters over the interval of time. In some embodiments, the various parameters may comprise an increase in a mean absolute value of the EMG signal, an increase in an amplitude of the EMG signal, an increase in a duration of muscle action potential in the EMG signal, a decrease in a frequency of the EMG signal, a change in the angular velocity signal, and a change in the acceleration signal. In some embodiments, the various parameters may further comprise a change in a ratio of an average value of the EMG signal to a stiffness of the object (200), a change in a ratio of the amplitude of the EMG signal to the stiffness of the object (200), a change in a ratio of the frequency of the EMG signal to the stiffness of the object (200), and a change in ratio of a duration of muscle action potential in the EMG signal to the stiffness of the object (200). In some embodiments, the stimulation may comprise a synchronous repetitive motion, an asynchronous repetitive motion, or a combination thereof. The motion sensor (100) may attach to, dangle from, or adhere to the body of the user.

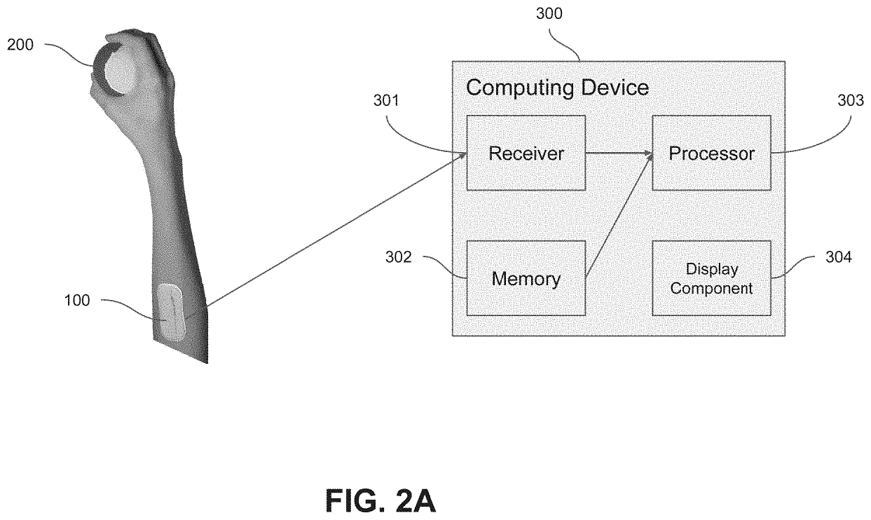

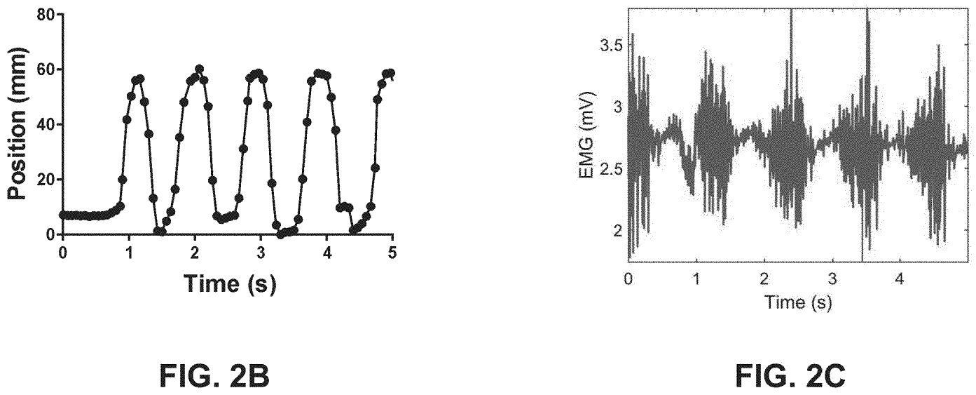

Referring now to FIG. 2A, the present invention features a system for measuring movements of one or more muscles of a body of a user in order to track muscular fatigue. In some embodiments, the system may comprise an object (200). The object (200) may be a squeezable ball or a weight. The system may further comprise a motion sensor (100). The motion sensor (100) may comprise an electromyogram (EMG) (101) for measuring an electrical activity in response to a stimulation of the one or more muscles of the body of the user in order to generate an EMG signal. The motion sensor (100) may further comprise a gyroscope (103) for measuring an angular velocity of the motion sensor (100) and the one or more muscles of the body of the user in response to the stimulation in order to generate an angular velocity signal. The motion sensor (100) may further comprise an accelerometer (102) for measuring an acceleration of the motion sensor (100) and the one or more muscles of the body of the user in response to the stimulation, and an orientation of the motion sensor (100) and the one or more muscles of the body of the user in relation to gravity to generate an acceleration signal and an orientation signal, respectively. The motion sensor (100) may further comprise a transmission component (104) for transmitting the EMG signal, the angular velocity signal, the acceleration signal, and the orientation signal to a computing device (300). The motion sensor (100) may further comprise a plurality of stretchable interconnects. Each stretchable interconnect (400) may extend between two components to connect said two components in order to facilitate conformal attachment to the subject. The stimulation may comprise an action carried out repetitively over an interval of time. In some embodiments, this action may be squeezing an object (200) in a hand, and squeezing the object (200) between thighs, or moving a back against the object (200). The motion sensor (100) is configured to measure a progressive reduction in a signal over the repetitive motions. The motion sensor (100) may be disposed relative to the one or more muscles of the body of the user.

The system may further comprise a computing device (300) comprising a display component (304). The computing device (300) may further comprise a memory component (302). The memory component (302) may comprise computer-readable instructions for generating a muscular fatigue signal by measuring various parameters. In some embodiments, the various parameters may comprise an increase in a mean absolute value of the EMG signal, an increase in an amplitude of the EMG signal, an increase in a duration of muscle action potential in the EMG signal, a decrease in a frequency of the EMG signal, a change in the angular velocity signal, and a change in the acceleration signal. The memory component (302) may further comprise instructions for displaying the muscular fatigue signal on the display component (304). The computing device (300) may further comprise a processor (303) capable of executing the computer-readable instructions stored on the memory component (302). The computing device (300) may further comprise a receiver component (301) for receiving the EMG signal, the angular velocity signal, the acceleration signal, and the orientation signal from the motion sensor (100).

In some embodiments, the various parameters may further comprise a change in a ratio of an average value of the EMG signal to a stiffness of the object (200), a change in a ratio of the amplitude of the EMG signal to the stiffness of the object (200), a change in a ratio of the frequency of the EMG signal to the stiffness of the object (200), and a change in ratio of a duration of muscle action potential in the EMG signal to the stiffness of the object (200). In some embodiments, the stimulation may comprise a synchronous repetitive motion, an asynchronous repetitive motion, or a combination thereof. In some embodiments, the system may further comprise a stress sensor disposed within the object (200) for measuring a force exerted on the object (200) by the stimulation, and an object transmission component disposed within the object (200) for transmitting the force measurement to the computing device (300). In some embodiments, the system may further comprise a visual tracking component capable of generating a body motion signature by measuring a displacement of the object (200) during the stimulation, a displacement of the one or more muscles of the body of the user, a time between repetitive motions of the stimulation, an acceleration of the stimulation, and a velocity of the stimulation. The body motion signature generated by the visual tracking component may be received by the computing device (300). The memory component (302) may further comprise instructions for generating the muscle fatigue signal by measuring a change in a time between repetitive motions of the stimulation, a change in a rate of the stimulation, and a change in a displacement of the object (200) during the stimulation.

In some embodiments, the memory component (302) may further comprise instructions for training a neural network with a plurality of diseased muscle fatigue signals, using the neural network to generate a comparison between the muscle fatigue signal to the plurality of diseased muscle fatigue signals, and diagnosing the user with a muscle disease based on the comparison. The plurality of diseased muscle fatigue signals may comprise physical biomarkers representing muscle frailty, hyperthyroid, kashiercore, and cancer. In some embodiments, the action may be an extension of the object (200) in the hand. The motion sensor (100) may attach to, dangle from, or adhere to the body of the user.

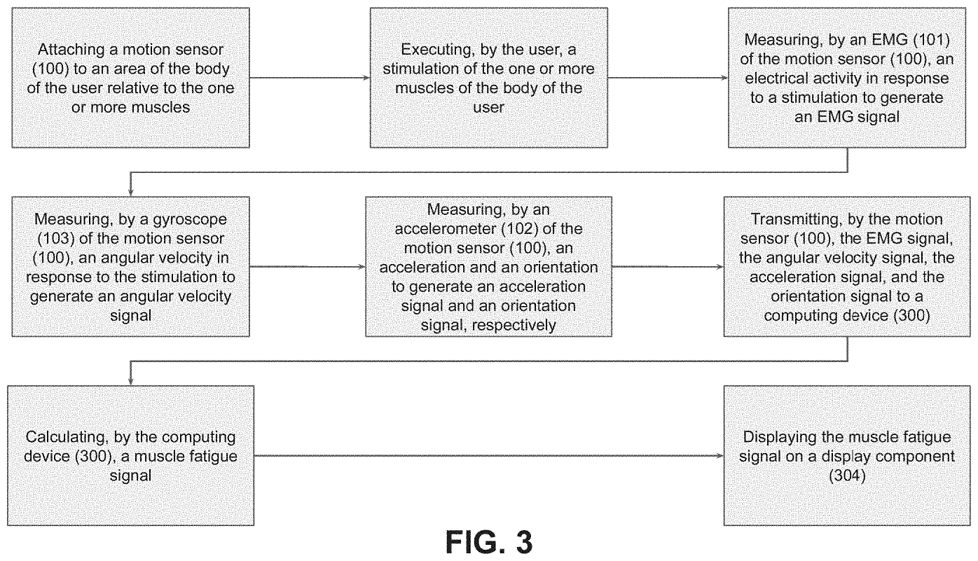

Referring now to FIG. 3, the present invention features a method for measuring movements of one or more muscles of a body of a user in order to track muscular fatigue. In some embodiments, the method may comprise attaching a motion sensor (100) to an area of the body of the user relative to the one or more muscles, and executing a stimulation of the one or more muscles of the body of the user. In some embodiments, the stimulation may comprise an action carried out repetitively over an interval of time. The action may be squeezing an object (200) in a hand, squeezing the object (200) between thighs, or moving a back against the object (200). The motion sensor (100) is configured to measure a progressive reduction in a signal over the repetitive motions. The method may further comprise using an EMG (101) of the motion sensor (100) to measure an electrical activity in response to a stimulation of the one or more muscles of the body of the user in order to generate an EMG signal. The method may further comprise using a gyroscope (103) of the motion sensor (100) to measure an angular velocity of the motion sensor (100) and the one or more muscles of the body of the user in response to the stimulation in order to generate an angular velocity signal. The method may further comprise using an accelerometer (102) of the motion sensor (100) to measure an acceleration of the motion sensor (100) and the one or more muscles of the body of the user in response to the stimulation, and an orientation of the motion sensor (100) and the one or more muscles of the body of the user in relation to gravity to generate an acceleration signal and an orientation signal, respectively. The method may further comprise transmitting the EMG signal, the angular velocity signal, the acceleration signal, and the orientation signal to a computing device (300). The method may further comprise calculating a muscle fatigue signal by measuring various parameters. The various parameters may comprise an increase in a mean absolute value of the EMG signal, an increase in an amplitude of the EMG signal, an increase in a duration of muscle action potential in the EMG signal, a decrease in a frequency of the EMG signal, a change in the angular velocity signal, and a change in the acceleration signal. The method may further comprise displaying the muscle fatigue signal on a display component (304).

In some embodiments, the various parameters may further comprise a change in a ratio of an average value of the EMG signal to a stiffness of the object (200), a change in a ratio of the amplitude of the EMG signal to the stiffness of the object (200), a change in a ratio of the frequency of the EMG signal to the stiffness of the object (200), and a change in ratio of a duration of muscle action potential in the EMG signal to the stiffness of the object (200). In some embodiments, the stimulation may comprise a synchronous repetitive motion, an asynchronous repetitive motion, or a combination thereof. In some embodiments, the method may further comprise steps for measuring, by a stress sensor disposed within the object (200), a force exerted on the object (200) by the stimulation, and transmitting the force measurement to the computing device (300). In some embodiments, the method may further comprise steps for generating, by a visual tracking component, a body motion signature by measuring a displacement of the object (200) during the stimulation, a displacement of the one or more muscles of the body of the user, a time between repetitive motions of the stimulation, an acceleration of the stimulation, and a velocity of the stimulation. The method may further comprise steps for receiving, by the computing device (300), the body motion signature, and generating the muscle fatigue signal by measuring a change in a time between repetitive motions of the stimulation, a change in a rate of the stimulation, and a change in a displacement of the object (200) during the stimulation,

In some embodiments, the method may further comprise steps for training a neural network with a plurality of diseased muscle fatigue signals, using the neural network to generate a comparison between the muscle fatigue signal to the plurality of diseased muscle fatigue signals, and diagnosing the user with a muscle disease based on the comparison. The plurality of diseased muscle fatigue signals may comprise physical biomarkers representing muscle frailty, hyperthyroid, kashiercore, and cancer. In some embodiments, the action may be an extension of the object (200) in the hand. In some embodiments, the object (200) is a squeezable ball or a weight. In some embodiments, the motion sensor (100) may attach to, dangle from, or adhere to the body of the user.

### EXAMPLE

The following is a non-limiting example of the present invention. It is to be understood that said example is not intended to limit the present invention in any way. Equivalents or substitutes are within the scope of the present invention.

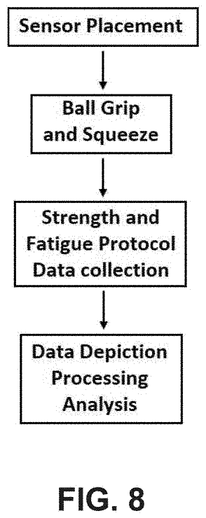

To study and analyze the proposed method amenable to routine “in-clinic” assessment of frailty, an experiment was designed, depicted in the flow chart shown in FIG. 8. As previous studies demonstrate that EMG fatigue signs in forearm extensor muscles were observed during motions such as gripping and compressing, a wearable sensor, BioStampRCT™ (MC10 Inc., Lexington, Mass., herein referred to as BioStamp), was taped to the volar surface of the forearm of each subject in order to observe and quantify muscle movement (extensor digitorum communis) during each test. Gyroscopic and EMG data were recorded within the BioStamp at sampling rate=250 Hz and uploaded to the cloud. A semaphore was placed on the second segment of the middle finger of the dominant hand of the subject. The motions of the semaphore were captured using a camera attached to a stand overlooking the hand, and were subsequently analyzed utilizing a video tracking software (Tracker, Open Source Physics). Ten healthy volunteers (8 M age 17-36, 2 F age 16-19) served as test subjects, in accordance with University of Arizona IRB (#1809925234) regulation, and were instructed to repeatedly compress a ball, while avoiding rotating their wrist, at a consistent comfortable rate for 1 minute, Enough time (10 min) in between trials was given so subjects would no longer feel the effects of fatigue resulting from the previous experiment. Finally, both BioStamp data and motion tracking data were quantitatively analyzed for frailty detection.

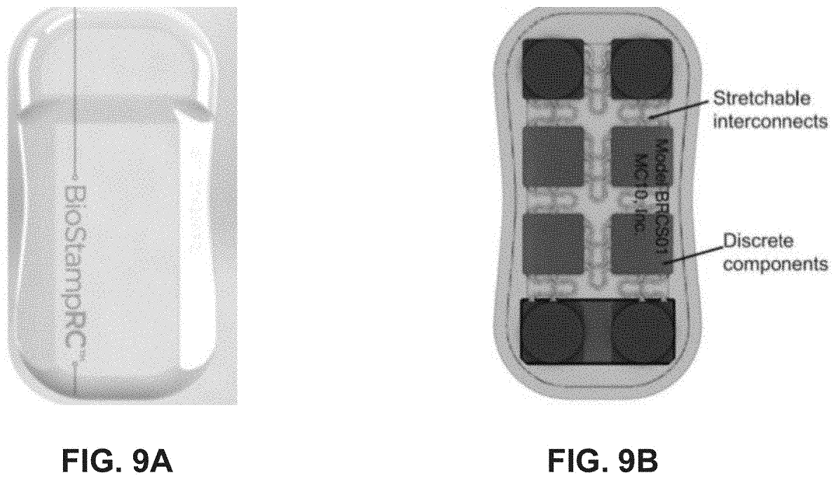

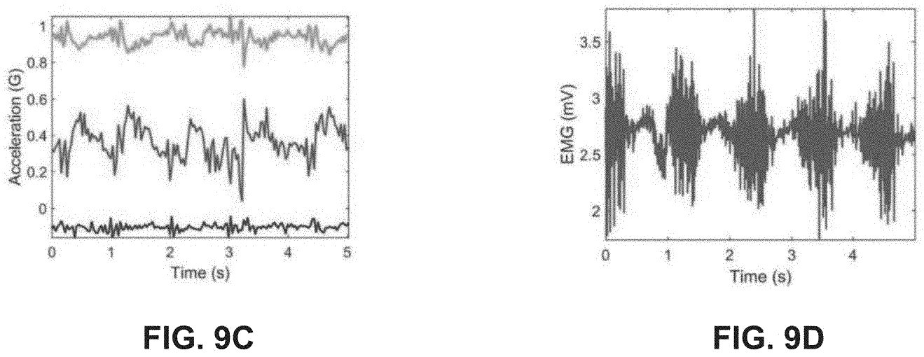

The wearable sensor BioStamp is a thin, pliable “Band Aid”-like device (FIGS. 9A-9B) directly applied to skin surface (FIG. 2A) and is controlled via Bluetooth from the MC10 Discovery App, pre-loaded on the tablet. Once a BioStamp is adhered to a subject, the sensor can be selected to start or stop recording EMG and accelerometer data (FIGS. 9C-9D) from the tablet and upload them via Bluetooth to the cloud where they can be accessed and downloaded from the MC10 Investigator Portal website.

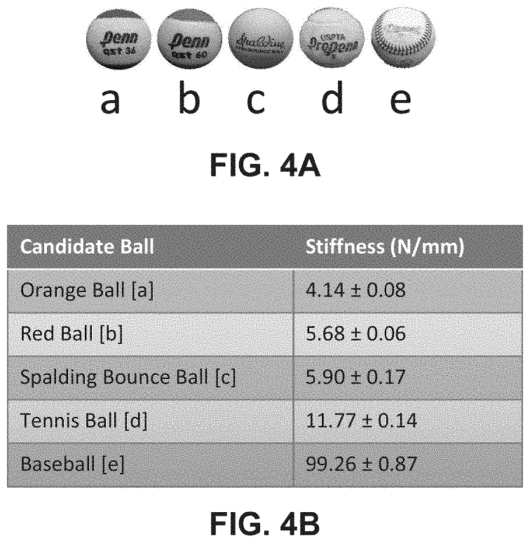

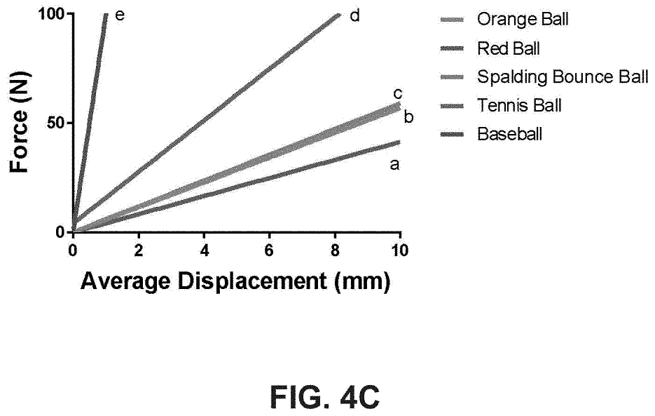

Five readily-accessible balls of varying stiffness (Penn QST 36, Penn QST 60, Spalding High-Bounce Ball, Pro Penn Marathon Tennis Ball, and Diamond DPL-1 Leather Baseball) were selected for compression testing. A controlled force was generated by a digital Tritest (ELE International 50 Load Frame, Bedfordshire, UK) and applied to each candidate ball at a constant increasing rate. Axial displacement was measured by the Tritest probe. The Young's module (stiffness) of each ball was then quantitatively acquired. The relative stiffness of candidate balls are shown in FIG. 4B.

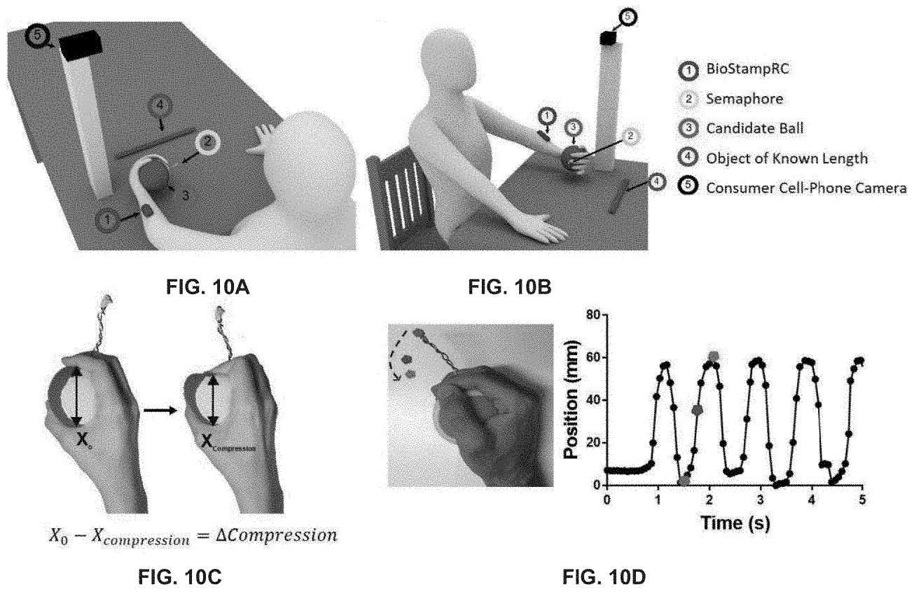

To quantitatively and accurately obtain and track the ball compression progress, a standard consumer grade cellphone (in this study, an iPhone7) was used, recording at 30 fps using a 12 MP rear camera. Subject would sit in front of a desk with the cellphone placed at an elevated height (FIGS. 10A-10B). The sequential cycles, tracking the complete excursion of the semaphore, from rest to complete compression and back to rest is presented in FIG. 100. The placement of the protruding semaphore was noted, along with the corresponding timestamp, for every frame of the video. The green maker represents the semaphore location corresponding to complete compression, in the shown motion capture and the peak of the position cycle. The red marker represents rest, in the shown motion capture and the through of the cycle in FIG. 10D. Compression was then defined as the peak-valley difference of the position of the semaphore.

The wearable sensor BioStamp allows continuous monitoring target muscle fatigue during the ball compression performance by the method of EMG. Biochemical and physiological changes during muscle fatiguing are reflected in properties of myoelectric signals captured on the surface of the skin above the target muscle. Comparing the activation to activation (spike to spike) time delay of muscle activity can define the muscle fatigue as a decrease in muscle action potential velocity. The motions captured by a consumer grade cell phone provide ball compression changes due to muscle fatigue. Comparing the peak to valley distance of semaphore movement also defines the muscle fatigue as a decrease in muscle strength.

Each compression test was repeated 3 times by each subject. All data from the BioStamp and motion capture were analyzed using JMP 13 (SAS Institute, North Carolina). Differences in fatigue and ball compression were statistically analyzed using Welch's t-test with statistical significance at p<0.05.

Two types of balls with distinctive different stiffness, Spalding Bouncing Ball (ball [c] for isotonic) and Baseball (ball [e] for isometric) here, were chosen to study the difference between isotonic and isometric muscle motion during ball compression to assess frailty.

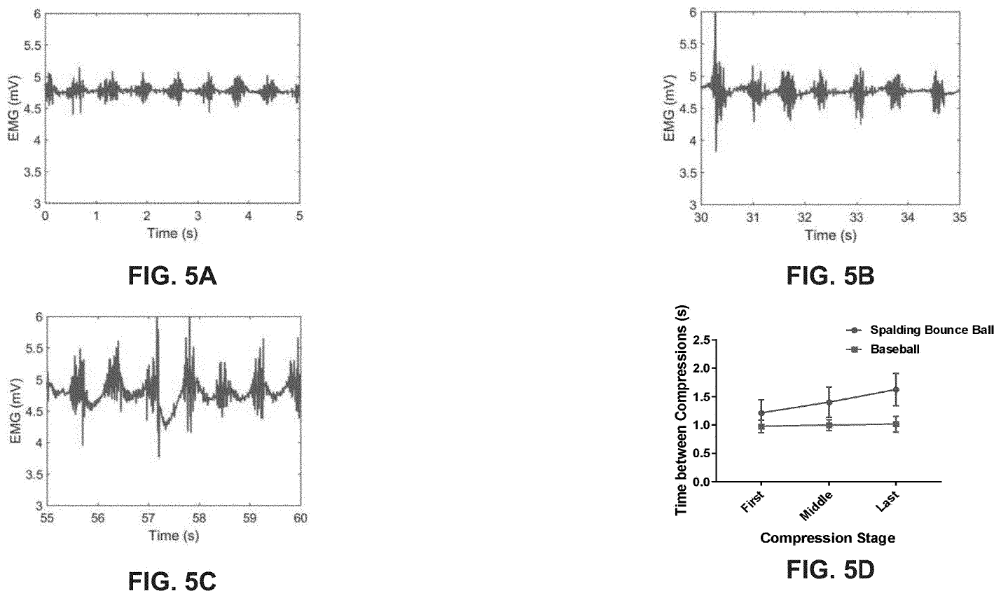

In light of proposing a simple and fast monitoring method amendable to routine “in-clinic” to assess frailty, raw EMG data was obtained, uploaded and processed by real-time software and specially adapted code to calculate the time interval between two consecutive muscle activations only. Longer delay in muscle activation time indicates muscle fatigue. FIGS. 5A-5C displays representative EMG measurements during the first, middle and last stages over a one-minute interval of isotonic and isometric compressions, respectively. Spikes in EMG voltage indicate myoelectric signals and therefore muscle activation. The changes in time between each EMG compression over time for isotonic and isometric compressions of all subjects are shown in FIG. 5D.

Noticeably, only the isotonic compression case showed the time between each muscle activation was gradually elongated during the 60 second ball compression assessment as one would anticipate. In contrast, volunteers were noted to have fairly minimal change in activation time delay or muscle fatigue during duration of the isometric compression case. As a comparison, slopes of activation time delay as volunteers were repeatedly conducting isotonic and isometric compressions were roughly 0.0075 and 0.00073 (mV/s), respectively.

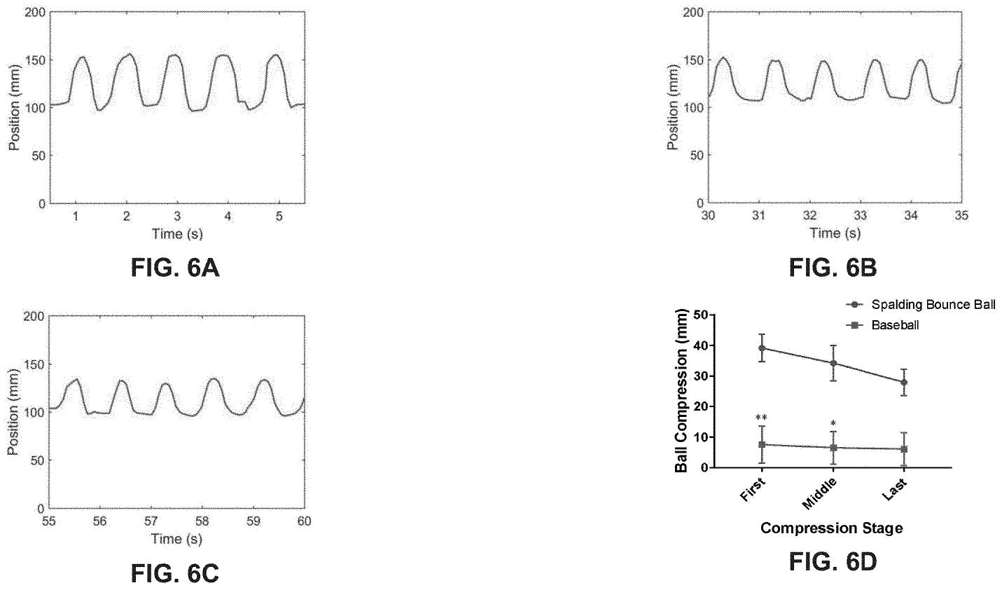

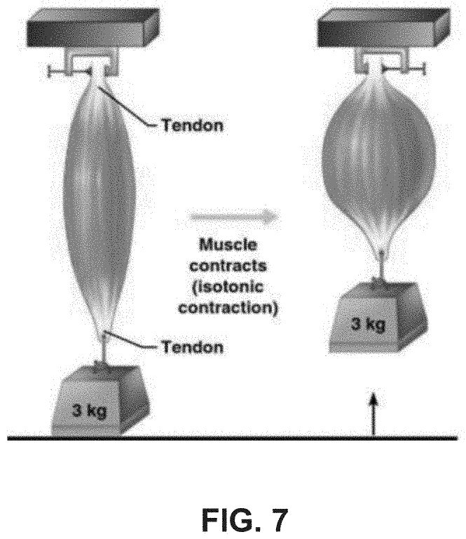

The motion tracking data of semaphore excursion during ball compression progress gives a more direct indication of muscle fatigue as a decrease in muscle strength. FIGS. 6A-60 display curves of semaphore position against time during first, middle and last stages of isotonic and isometric compressions, respectively. Position difference represents the motion of semaphore and is a reflection of ball compression movement. The changes in ball compression over time for isotonic and isometric compressions of all subjects are shown in FIG. 6D.

As expected, the isometric compression illustrated fairly flat trajectories throughout the whole assessment due to the fact that baseball is with high stiffness. Slight compressions were still observed, which is largely due to low stiffness and high compliance of subjects' hands. The isotonic compression case showed the change of compression more clearly and illustrated the compression distance between each muscle activation was gradually diminished during the 60 second ball compression assessment. Volunteers were noted to have compressed a Spalding bounce roughly 29% less after 60 seconds, while that of a baseball was 20%, on average.

Frailty detection and assessment is crucial for intervention decision making for frail patients, especially for older adults as they suffer from low physiologic reserve as aging and are more vulnerable to risk of adverse health outcomes, such as mortality, institutionalization, falls, and hospitalization. Current clinical setups normally relies on bulky and time-consuming subjective questionnaires or requires spatial motion that is not applicable for mobility-impaired nonambulatory patients. A critical obstacle to the success of effectively target care is the absence of simple and fast approaches to identify and assess frailty. Therefore, a need exists to provide access to simple, mobile and inexpensive systems for accurate and comprehensive assessment of frailty in an uncontrolled non-laboratory environment (e.g. quick clinic or self at home). This study introduced the utility of a conformal, wireless, wearable patch system, along with motion tracking from a consumer grade cellphone, to obtain muscle activation information for assessment of frailty. With this system, we were able to collect necessary motion information over time to quantify muscle fatigue status, without the necessity of tethering to expensive external equipment.

The utilization of both EMG sensor and video motion tracking during ball compression activity in the proposed system allows for characterization of upper-extremity muscle in both spatial and time spaces. Although the BioStamp is also equipped with accelerometers and gyroscopes, providing the ability to obtain 3-dimensional accelerations and angular velocities to reconstruct 3D motion trajectories, accelerometers and gyroscopes commonly are plagued with error accumulation and value-reading drift when integrating for spatial displacement in small motion scale. Therefore, only EMG data was used to acquire muscle fatigue status. Though a large number of studies have been performed on different signal processing methods and techniques to EMG signals in fatigue-inducing situations and a good review is provided by Cifrek. et al., in light of developing a simple and fast monitoring method amendable to routine “in-clinic” to assess frailty, only raw EMG data was obtained, uploaded and processed.

As the integrated accelerometers and gyroscopes are not suitable for providing accurate readings in small-scale motion, visual tracking utilizing cameras or markers placed on the human body was used here for hand motion capture. As subjects comfortably lay their hand on a table to perform the ball compression test as shown in FIGS. 10A-10B, the bottom side of palm (not sure what the correct term is) grants a steady hand orientation when subject is performing tasks such as compression or gripping, which allowing accurate planar trajectory tracking.

As observed, continuous ball compression indeed led to measurable reduced muscle strength and lowered motion speed, which are markers of frailty. Isotonic compression, requiring the muscle both contracts and changes its length, demonstrated clear changes in both ball compression distance and muscle activation to activation time. The results from volunteers showed, on average, the gradual delayed muscle activation time was 0.41 second (almost 34% increase) after a 60-second assessment. This muscle action potential velocity decrease is an obvious indicator of muscle fatigue. Similarly, all of the volunteers demonstrated muscle strength diminished about 29% after 60 seconds even though they are instructed to compress balls as consistent as possible.

Contrast to intuitive thinking, isometric compression did not show significant variations of motion speed (only 0.04 second reduced, about 4%), which might be explained by psychological factors such as lack of motivation or task and goal condition, Though minimal, all volunteers showed reduced muscle strength after a 60-second isometric ball compression activity, about 20%.

A fast and simple method amenable to assess frailty in clinic using a combination of wireless, stretchable sensors and consumer grade cellphones showed adequacy for frailty assessment in un-controlled and limited environments. The proposed method does not depend on cumbersome machines and time-consuming questionnaires, It requires very minimal space to perform and utilizes common accessible objects, which extends the applicability to non-ambulatory patients.

Muscle fatigue can be tracked accurately, and reliably using only a cell phone and a BioStampRC in a point-of-care solution. The diminution of the compression over time shows that a phone can track loss of strength due to fatigue. The BioStampRC's EMG measurement provides enough data to quantify the compression cycle. These elements can be combined to create a comprehensive analysis of muscle fatigue. In the future, this proposed method will be extended to include more volunteers, healthy ones and patients, and also validate the results with the well-established frailty quantification standard, Rockwood questionnaire.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims, In some embodiments, the figures presented in this patent application are drawn to scale, including the angles, ratios of dimensions, etc. In some embodiments, the figures are representative only and the claims are not limited by the dimensions of the figures, In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings,

1. A motion sensor (100) for measuring movements of one or more muscles of a body of a user in order to track muscular fatigue, the motion sensor (100) comprising:
a. an electromyogram (EMG) (101) for measuring an electrical activity in response to a stimulation of the one or more muscles of the body of the user in order to generate an EMG signal;
b. a gyroscope (103) for measuring an angular velocity of the motion sensor (100) and the one or more muscles of the body of the user in response to the stimulation in order to generate an angular velocity signal;
c. an accelerometer (102) for measuring an acceleration of the motion sensor (100) and the one or more muscles of the body of the user in response to the stimulation, and an orientation of the motion sensor (100) and the one or more muscles of the body of the user in relation to gravity to generate an acceleration signal and an orientation signal, respectively;
d. a transmission component (104) for transmitting the EMG signal, the angular velocity signal, the acceleration signal, and the orientation signal to a computing device (300); and
e. a plurality of stretchable interconnects, wherein each stretchable interconnect (400) extends between two components to connect said two components in order to facilitate conformal attachment to the subject;
wherein the stimulation comprises an action carried out repetitively over an interval of time;
wherein the motion sensor (100) is disposed relative to the one or more muscles of the body of the user; and
wherein the computing device (300) is capable of measuring muscular fatigue by measuring over the interval of time:
i. an increase in a mean absolute value of the EMG signal,
ii. an increase in an amplitude of the EMG signal,
iii. an increase in a duration of muscle action potential in the EMG signal,
iv. a decrease in a frequency of the EMG signal,
v. a change in the angular velocity signal,
vi. a change in the acceleration signal, and
vii. a change in a ratio of an average value of the EMG signal to a stiffness of the object (200).

2. (canceled)

3. The motion sensor (100) of claim 1, wherein the computing device (300) is capable of measuring over the interval of time:
a. a change in a ratio of the amplitude of the EMG signal to the stiffness of the object (200);
b. a change in a ratio of the frequency of the EMG signal to the stiffness of the object (200); and
c. a change in ratio of a duration of muscle action potential in the EMG signal to the stiffness of the object (200).

4. (canceled)

5. The motion sensor (100) of claim 1, wherein the stimulation comprises a synchronous repetitive motion, an asynchronous repetitive motion, or a combination thereof.

6. The motion sensor (100) of claim 1, wherein the motion sensor (100) attaches to, dangles from, or adheres to the body of the user.

7. A system for measuring movements of one or more muscles of a body of a user in order to track muscular fatigue, the system comprising:
a. an object (200);
b. a motion sensor (100) comprising:
i. an EMG (101) for measuring an electrical activity in response to a stimulation of the one or more muscles of the body of the user in order to generate an EMG signal,
ii. a gyroscope (103) for measuring an angular velocity of the motion sensor (100) and the one or more muscles of the body of the user in response to the stimulation in order to generate an angular velocity signal,
iii. an accelerometer (102) for measuring an acceleration of the motion sensor (100) and the one or more muscles of the body of the user in response to the stimulation, and an orientation of the motion sensor (100) and the one or more muscles of the body of the user in relation to gravity to generate an acceleration signal and an orientation signal, respectively,
iv. a transmission component (104) for transmitting the EMG signal, the angular velocity signal, the acceleration signal, and the orientation signal to a computing device (300),
v. a plurality of stretchable interconnects, wherein each stretchable interconnect (400) extends between two components to connect said two components in order to facilitate conformal attachment to the subject,
wherein the stimulation comprises an action carried out repetitively over an interval of time,
wherein the action is selected from a group comprising squeezing an object (200) in a hand, squeezing the object (200) between thighs, and moving a back against the object (200),
wherein the motion sensor (100) is disposed relative to the one or more muscles of the body of the user; and
c. the computing device (300) comprising:
i. a display component (304),
ii. a memory component (302) comprising computer-readable instructions for:
1. generating a muscular fatigue signal by measuring:
A. an increase in a mean absolute value of the EMG signal,
B. an increase in an amplitude of the EMG signal,
C. an increase in a duration of muscle action potential in the EMG signal,
D. a decrease in a frequency of the EMG signal,
E. a change in the angular velocity signal, [and]
F. a change in the acceleration signal; and
G. a change in a ratio of an average value of the EMG signal to a stiffness of the object (200); and
2. displaying the muscular fatigue signal on the display component (304),
iii. a processor (303) capable of executing the computer-readable instructions stored on the memory component (302), and
iv. a receiver component (301) for receiving the EMG signal, the angular velocity signal, the acceleration signal, and the orientation signal from the motion sensor (100).

8. The system of claim 7, wherein the memory component (302) further comprises instructions for measuring over the interval of time:
a. a change in a ratio of the amplitude of the EMG signal to the stiffness of the object (200);
b. a change in a ratio of the frequency of the EMG signal to the stiffness of the object (200); and
c. a change in ratio of a duration of muscle action potential in the EMG signal to the stiffness of the object (200).

9. The system of claim 7, wherein the stimulation comprises a synchronous repetitive motion, an asynchronous repetitive motion, or a combination thereof.

10. The system of claim 7 further comprising:
a. a stress sensor disposed within the object (200) for measuring a force exerted on the object (200) by the stimulation; and
b. an object transmission component disposed within the object (200) for transmitting the force measurement to the computing device (300).

11. The system of claim 7 further comprising a visual tracking component capable of generating a body motion signature by measuring:
a. a displacement of the object (200) during the stimulation;
b. a displacement of the one or more muscles of the body of the user;
c. a time between repetitive motions of the stimulation;
d. an acceleration of the stimulation; and
e. a velocity of the stimulation.

12. The system of claim 11, wherein the body motion signature is received by the computing device (300) and the memory component (302) further comprises instructions for:
a. generating the muscle fatigue signal by measuring:
i. a change in a time between repetitive motions of the stimulation,
ii. a change in a rate of the stimulation, and
iii. a change in a displacement of the object (200) during the stimulation.

13. The system of claim 7, wherein the memory component (302) further comprises instructions for:
a. training a neural network with a plurality of diseased muscle fatigue signals;
b. using the neural network to generate a comparison between the muscle fatigue signal to the plurality of diseased muscle fatigue signals; and
c. diagnosing the user with a muscle disease based on the comparison.

14-16. (canceled)

17. The system of claim 7, wherein the motion sensor (100) attaches to, dangles from, or adheres to the body of the user.

18. A method for measuring movements of one or more muscles of a body of a user in order to track muscular fatigue, the method comprising:
a. attaching a motion sensor (100) to an area of the body of the user relative to the one or more muscles;
b. executing, by the user, a stimulation of the one or more muscles of the body of the user,
wherein the stimulation comprises an action carried out repetitively over an interval of time,
wherein the action is selected from a group comprising squeezing an object (200) in a hand, squeezing the object (200) between thighs, and moving a back against the object (200),
c. measuring, by an EMG (101) of the motion sensor (100), an electrical activity in response to a stimulation of the one or more muscles of the body of the user in order to generate an EMG signal;
d. measuring, by a gyroscope (103) of the motion sensor (100), an angular velocity of the motion sensor (100) and the one or more muscles of the body of the user in response to the stimulation in order to generate an angular velocity signal;
e. measuring, by an accelerometer (102) of the motion sensor (100), an acceleration of the motion sensor (100) and the one or more muscles of the body of the user in response to the stimulation, and an orientation of the motion sensor (100) and the one or more muscles of the body of the user in relation to gravity to generate an acceleration signal and an orientation signal, respectively;
f. transmitting, by the motion sensor (100), the EMG signal, the angular velocity signal, the acceleration signal, and the orientation signal to a computing device (300);
g. calculating, by the computing device (300), a muscle fatigue signal by measuring over the interval of time:
i. an increase in a mean absolute value of the EMG signal,
ii. an increase in an amplitude of the EMG signal,
iii. an increase in a duration of muscle action potential in the EMG signal,
iv. a decrease in a frequency of the EMG signal,
v. a change in the angular velocity signal, [[and]]
vi. a change in the acceleration signal; and
vii. a change in a ratio of an average value of the EMG signal to a stiffness of the object (200); and
h. displaying the muscle fatigue signal on a display component (304).

19. The method of claim 18 further comprising steps for measuring over the interval of time:
a. a change in a ratio of the amplitude of the EMG signal to the stiffness of the object (200);
b. a change in a ratio of the frequency of the EMG signal to the stiffness of the object (200); and
c. a change in ratio of a duration of muscle action potential in the EMG signal to the stiffness of the object (200).

20. The method of claim 18, wherein the stimulation comprises a synchronous repetitive motion, an asynchronous repetitive motion, or a combination thereof.

21. The method of claim 18 further comprising steps for:
a. measuring, by a stress sensor disposed within the object (200), a force exerted on the object (200) by the stimulation; and
b. transmitting the force measurement to the computing device (300).

22. The method of claim 18 further comprising steps for:
a. generating, by a visual tracking component, a body motion signature by measuring:
i. a displacement of the object (200) during the stimulation;
ii. a displacement of the one or more muscles of the body of the user;
iii. a time between repetitive motions of the stimulation;
iv. an acceleration of the stimulation; and
v. a velocity of the stimulation.

23. The method of claim 22 further comprising steps for:
a. receiving, by the computing device (300), the body motion signature; and:
b. generating the muscle fatigue signal by measuring:
i. a change in a time between repetitive motions of the stimulation,
ii. a change in a rate of the stimulation, and
iii. a change in a displacement of the object (200) during the stimulation.

24. The method of claim 18 further comprising steps for:
a. training a neural network with a plurality of diseased muscle fatigue signals;
b. using the neural network to generate a comparison between the muscle fatigue signal to the plurality of diseased muscle fatigue signals; and
c. diagnosing the user with a muscle disease based on the comparison.

25-27. (canceled)

28. The method of claim 18, wherein the motion sensor (100) attaches to, dangles from, or adheres to the body of the user.
